(19) United States
(12) Plant Patent Application Publication
(10) Pub. No.: US 2007/0199115 P1
(43) Pub. Date: Aug. 23, 2007

(54) Floribunda rose plant named 'Poulcas023'

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/360,399

(22) Filed: Feb. 22, 2006

(51) Int. Cl. A01H 5/00

(52) U.S. Cl. PLT/145

(57) ABSTRACT

A new garden rose plant of the floribunda class which has abundant, yellow-orange flowers and attractive foliage. This new and distinct variety has shown to be uniform and stable in the resulting generations from asexual propagation.

### BOTANICAL DESIGNATION

Rosa hybrid

### VARIETY DENOMINATION

‘Poulcas023’

### SUMMARY OF THE INVENTION

The present invention constitutes a new and distinct variety of garden rose plant which originated from a controlled crossing between the female seed parent ‘Poulfl002’, described and illustrated in U.S. Plant Pat. No. 15,053, and the male pollen parent, an unnamed seedling.

The two parents were crossed during the summer of 1995 and the resulting seeds were planted in a controlled environment in Fredensborg, Denmark. The new variety, named ‘Poulcas023’, originated as a single seedling from the stated cross.

The new variety may be distinguished from its female seed parent by the following combination of characteristics:
1. The claimed plant has yellow-orange flowers while the seed parent has pink flowers.
2. The seed parent has 20 to 25 petals. ‘Poulcas023’ has 65-75 petals.

The new variety may be distinguished from its male pollen parent primarily by flower color. The pollen parent has solid pink flowers, while the claimed plant has yellow-orange flowers.

The objective of the hybridization of this rose variety was to create a new and distinct variety for garden use with unique qualities, such as:
1. Uniform and abundant yellow-orange flowers;
2. Vigorous, but compact and uniform growth when propagated both as a budded rose and on its own roots;
3. Exceptional disease resistance;
4. Suitability for cultivation within small containers.

This combination of qualities is not present in previously available commercial cultivars of this type, known to the inventor, and distinguish ‘Poulcas023’ from all other varieties of which we are aware.

As part of the rose development program, Mogens N. Olesen germinated the seeds from the aforementioned hybridization during winter of 1995 and conducted evaluations on the resulting seedlings in a controlled environment in Fredensborg, Denmark. ‘Poulcas023’ was selected in the spring of 1996 by the inventor as a single plant from the progeny of the aforementioned hybridization.

Asexual reproduction of ‘Poulcas023’ by traditional budding and rooted cuttings was first done by Mogens N. Olesen in the nursery in Fredensborg, Denmark in July, 1996. This initial and other subsequent asexual propagations conducted in controlled environments have demonstrated that the characteristics of ‘Poulcas023’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulcas023’. Specifically illustrated in the drawing are:

FIG. 1.1; Flower bud closed, flower bud as sepals unfold, and partially open;

FIG. 1.2; Open flowers;

FIG. 1.3; Sepals, receptacle, and peduncle;

FIG. 1.4; Mature leaf;

FIG. 1.5; Flower petals, detached;

FIG. 1.6; Bare stem exhibiting prickles;

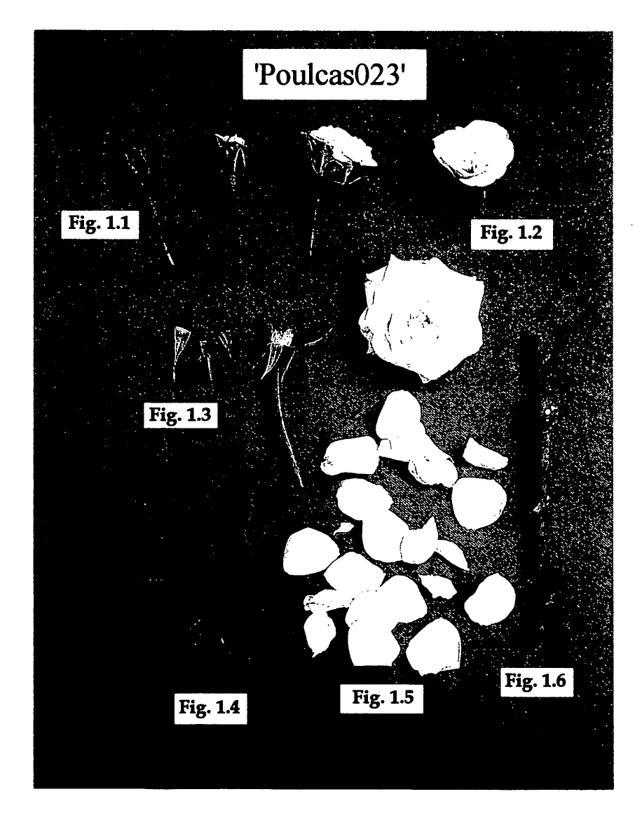

### DETAILED DESCRIPTION OF THE VARIETY

The following is a description of ‘Poulcas023’, as observed in its growth in in a field nursery in Odense, Denmark. Observed plants are 2 years of age, and were grown on Rosa multiflora understock. Color references are made using The Royal Horticultural Society (London, England) Colour Chart, 2001, except where common terms of color are used.

For a comparison, several physical characteristics of the rose variety ‘Poulcs011’, U.S. Plant Pat. No. 15,232, are compared to ‘Poulcas023’ in Chart 1.

CHART 1

| | ‘Poulcas023’ | ‘Poulcs011’ |
|---|---|---|
| Petal Count | 65 to 75, normally 10 of | 35, with 9 petaloids |
| | which are petaloids | |
| Flower Diameter | 55-75 mm | 60 to 70 mm |
| Outer petals upon | Orange Group 27C at petal | Orange Group 27A |
| opening: upper | margin, blending to become | |
| petal surface color | Yellow Group 11C at the | |
| | middle zone. At the basal | |
| | zone petals change from | |
| | Yellow 12C to 12A at the | |
| | point of attachment. | |

### FLOWER AND FLOWER BUD

Blooming habit: Continuous.
Flower bud:
   Size.—Upon opening, 23 mm in length from base of receptacle to end of bud. Bud diameter is 14 mm.
   Bud form.—Ovate.
   Bud color.—As sepals unfold, petals are Orange Group 26A with intonations of Orange-Red Group 34A. And splotches of Greyed-Purple Group 183A to Red Group 53A observed.
   Sepals.—Upper Surface: Color: Yellow-Green Group 146B. Texture: Weak to medium pubescence observed. Lower Surface: Color: Yellow-Green Group 1446A with occasional anthocyanic pigments of Greyed-Purple Group 183A. Texture: Smooth. Shape: Apex: Cirrhose. Base: Flat at union with receptacle. Margins: Margins have medium foliaceous appendages on three of the five sepals. Stipitate glands are sparse. Size: 30 mm long by 7 mm wide.
   Receptacle.—Surface Texture: Smooth. Shape: Urn shaped. Size: 7 mm (h)×7 mm (w). Color: Yellow-Green Group 144A. Anthocyanin: Greyed-Purple Group 183A to 187B.
   Peduncle.—Surface: Rough. Length: 55 to 80 mm average length. Diameter: 3 mm. Color: Yellow-Green Group 145A. Anthocyanic pigments of Greyed-Purple 187B. Strength: Strong strength.
   Borne.—Flowers are borne in clusters of 3 to 7 buds per flowering stem on average. Inflorescence is a Corymb.
Flower bloom:
   Fragrance.—Medium rose scent.
   Duration.—As a garden plant, flowers last up to 15 to 20 days. Petals do not fall cleanly away from plant.
   Size.—55 to 75 mm. Average flower dept is 25 mm when open.
   Form.—Younger flower: Quartered rosette, double with many overlapping petals packed into quarter sections. Older flower: Rosette, double flower with many slightly overlapping petals of different sizes.
   Shape of flower, side view.—Upon opening: The upper portion is flat. The lower portion is flattened convex. After opening: The upper portion is convex. The lower portion is concave.
   Petalage.—Under normal conditions, flowers have 65 to 75 petals, normally 10 of which are petaloids.
   Petal color.—Upon opening, outer whorl of flower petals Upper surface: Yellow-Orange Group 22A with an overlay of Orange-Red Group 34A. Lower surface Yellow-Orange Group 22A with an overlay of Orange-Red Group 34A. Petal color streaks Greyed-Purple Group 183A observed. Upon opening, petal colors Upper surface: Orange Group 27C at petal margin, blending to become Yellow Group 11C at the middle zone. At the basal zone petals change from Yellow 12C to 12A at the point of attachment. Lower surface: 3 to 4 mm of petal margins are colored Red Goup 56A to 56B. Progressing towards the petal base, Yellow-Orange Group 19A to 19B at the middle zone. Basal zone is Orange Group 26A. Intonations of Yellow Group 6C radiate from the petal base, extending to the middle zone. After opening, inner and outer petals Upper surface: Red Group 56A to 56B at the apex, blending to become Red Group 56D. At the middle zone, coloration is Yellow Group 11C. Light streaks of Yellow-Orange Group 19B observed at middle zone. The basal zone is primarily Yellow Group 6A. Occasionally, the upper surface of outer petals exhibits color spots, Red-Purple Group 58B. Lower surface: Red Group 56A to 56B at the margin of the apex, blending to become Red Group 56D. At the middle zone, Yellow Group 11C with streaks of Yellow-Orange Group 19A and 19B radiating from the basal zone. The basal zone is Yellow 6A to 6C.
General tonality: On open flower Yellow-Orange Group 19C to 19D with margins colored Red Group 36B to 36C.
Petals:
   Petal reflex.—Somewhat reflexed at margins.
   Petal margin.—Entire and uniform.
   Shape.—Inner petals: Narrow elliptical. Outer petals: Broad elliptical. Base: Inner petals: Acute. Outer petals: Obtuse. Apex: Orbicular.
   Size.—Petal size varies. Outer petals 30 mm (l)×20 mm (w). Inner petals 40 mm (l)×43 mm (w).
   Texture.—Smooth.
   Thickness.—Average.
Petaloids:
   Quantity.—On average there are 10.
   Size.—15 mm long×10 mm wide.
   Shape.—Irregular and irregular, asymmetrical.
   Color.—The upper and undersurface are Yellow Group 10B to 10C at apex with intonations of Yellow-Orange Group 18A, and Yellow Group 6B at base.
Reproductive organs:
   Pollen.—None observed.
   Anthers.—Size: Normally 2.5 mm long. Color: Yellow-Orange Group 22A at margins. Yellow Group 10D at center. Quantity:
   Filaments.—Color: Yellow-Group 7A to 7B. Length: 6 to 7 mm.
   Pistils.—Length: Normally 5 mm long. Quantity: 85 to 95.
   Stigmas.—Inferior in relative to the length of the filaments and the height of the anthers. Color: Yellow Group 4B.
   Styles.—Color: Yellow Group 2D.
   Seed formation.—Not observed.

### PLANT

Plant growth: Upright, bushy, moderately compact. The average height of the plant itself is 60 to 100 cm.
Stems:
   Color.—Young wood: Yellow-Green Group 146A. Older wood: Yellow-Green Group 146A with anthocyanin the color of Greyed-Orange 176A.
   Internodal distance.—On mature canes, 50 mm.
   Length of stems.—On average, canes are 30 cm from the base of the stem to the flowering portion.
   Diameter.—9 mm.
   Surface texture.—Young wood: Smooth. Older wood: Smooth.
Prickles:
   Incidence.—3 prickles per 10 cm of stem.
   Length.—5 to 7 mm.
   Shape.—Upper side: Convex to flat. Lower side: Concave.
   Color.—Mature wood: Greyed-Yellow Group 160A to 160B.
Plant foliage: Normal number of leaflets on normal leaves in middle of the stem: 5 leaflets.
   Compound leaf size.—130 mm (l)×95 mm (w).
   Quantity.—2 leaves per 10 cm of stem.
   Color.—Mature foliage: Upper Leaf Surface: Yellow-Green Group 147A. Lower Leaf Surface: Yellow-Green Group 146B.
Plant leaves and leaflets:
   Stipules.—Size: 20 mm in length. Shape: Linear, slightly broad based with outward extending apecies. Margins: Finely serrated with few stipitate glands. Color: Green Group 143A.
   Petiole.—Length: 20 to 27 mm. Diameter: Normally 2 mm. Upper surface: Color: Yellow-Green Group 146A with intonations of Greyed-Purple Group 184A. Lower surface: Color: Yellow-Green Group 146B. Observations: Few small thorns below.
   Rachis.—Size: 45 to 50 mm in length. Upper surface: Color: Yellow-Green Group 146A with anthocyanic pigments of Greyed-Purple Group 184A. Lower surface: Color: Yellow-Green Group 146B. Observations: Few small glands on the upper side and a few small prickles below observed.
   Leaflet.—Size: 50 to 70 mm in length by 35 to 40 mm wide. Edge: Serrated. General Shape: Ovate. Apex Shape: Acuminate. Base Shape: Obtuse to rounded. Texture: Rugose. Thickness: Average. Arrangement: Odd pinnate. Venation: Reticulate. Leaf Gloss: Moderately glossy.
   Disease resistance.—Above average resistance to powdery and downy mildew, rust, black spot, and Botrytis under normal growing conditions in Jackson County, Oregon.
   Cold hardiness.—The variety is tolerant to USDA Cold Hardiness Zone 6.
   Heat tolerance.—The variety has been found to be suitable for climate conditions found in the American Horticulture Society heat zone 7.

1. A new and distinct variety of rose plant of the floribunda rose class named ‘Poulcas023’, substantially as illustrated and described herein, due to its abundant yellow-orange flowers, disease resistance, and extended period of bloom.
